SEQUENCE LISTING (60 sequences)

SEQ ID NO: 1 - DNA, 100 bp, Agrobacterium tumefaciens
cggtttttcgcgcacttgccttgtcccgaggggatgaggcatagtccgcgaatcaaaaacggcctgttccgtttcggggaagcgcacgattccgttgccg

SEQ ID NO: 2 - DNA, 100 bp, Agrobacterium tumefaciens
ggtcttgccgatgaggccaaggccaatctcggctccggcgttgtgttgctgattgcggtttctgacgatggcaaggcaagcgccgtcgctgccgtgaccg

SEQ ID NO: 3 - DNA, 100 bp, Agrobacterium tumefaciens
atgactgaacttgataccttgaaatcgcaactgatgtcggagatcgccgccgctgccgatgagccggccatcgaggccgtgcgcgtctccgcgctcggca

SEQ ID NO: 4 - DNA, 100 bp, Agrobacterium tumefaciens
acgcgctcgagaccctcgatctcgtgaaagagaaccgcgaaaaggtcgatttcagtcgaattaccgtcgaatccccgcgtgatctcagccatggcgatgt

SEQ ID NO: 5 - DNA, 100 bp, Agrobacterium tumefaciens
caagccatggccgcgaggactttgatgcctggacgaatagcgtccgcacccttgaagcacgcgggatcgataccaaaatcccgttcccggtcgatgacgc

SEQ ID NO: 6 - DNA, 100 bp, Agrobacterium tumefaciens
aacttcgagaccggtcaggcgcttcacttccgacagcggaaaatccacgaccttgggctgatgccccttgtaaccgacgatcttggcttcgccagcgatg

SEQ ID NO: 7 - DNA, 100 bp, Agrobacterium tumefaciens
agggtgcgggtcttggcgatgaacagatcgaaaaagtgctgttcttcgttggtatcaaggattatgcggcaagcgctgatgatctcgcaaaactggttgc

SEQ ID NO: 8 - DNA, 100 bp, Agrobacterium tumefaciens
agacctcgcgcaatatctggaacctgatggccgatagcggcgacatctacaaggacagctatgccggctggtattccgtgcgcgacgaggcctattacgc

SEQ ID NO: 9 - DNA, 100 bp, Agrobacterium tumefaciens
ttcaaggaggaggcccgcaagctaatgacgatcgacatgatcgaggacaatatcacctcgctcaagcacgtctttgcgaattacctcgattacgatcgtg

SEQ ID NO: 10 - DNA, 100 bp, Agrobacterium tumefaciens
atggcgcagaagaacatggaactggcggaaaagctgaaagccgaagtcgctgatatcaaggaaaacatgccgcgcgccgaggaggaggaccgtcaggtct

SEQ ID NO: 11 - DNA, 100 bp, Agrobacterium tumefaciens
catccgcatcgacggtctagtcaaggcgcgttcggaagacaccatcaacaaaggcatggcgaccggcgagatcgagctttacgcgcaggagatcgaagtt

SEQ ID NO: 12 - DNA, 100 bp, Agrobacterium tumefaciens
acatcgatttcggcggccatattcgcaacgccggtctgggcgttaatatcacggttggcgatgagccggtgcacgcttatttcccgatggtggagccgct

SEQ ID NO: 13 - DNA, 100 bp, Agrobacterium tumefaciens
ccggttgcgggatgagctttccgagaagggtattcagctgaaggatggcaaggacaaggagactggggaacggacgacgacgtgggagttgaagcggtag

SEQ ID NO: 14 - DNA, 100 bp, Agrobacterium tumefaciens
accgtcaacattcgccgtctcggttcgcagaaccagacgtcgatgtcgctcgaagaggcgatcaccagcctcgtggacgaggcgacgccgccggatgtca

SEQ ID NO: 15 - DNA, 100 bp, Agrobacterium tumefaciens
cgctcctttcgatgtcgtgatcatcaacatgaaggctggcgatgcggcctgcgatgcggcttgtgaaaagctgtattatcagctctccaacgccggcaag

SEQ ID NO: 16 - DNA, 100 bp, Agrobacterium tumefaciens
ccgaagccggcgaccgaaggcgaagggctggcggcctccaaggcagcccacaagacgctgaaggctgttcaggaagatctggacaagctcgccttcaaca

SEQ ID NO: 17 - DNA, 100 bp, Agrobacterium tumefaciens
gacttcaacgatttcgaagtcggcaagcgttgcggcctgcgcgccatcaacgtcatgaacatcgatggtacgatctccatcaaggaaaacgaagatttcc

SEQ ID NO: 18 - DNA, 100 bp, Corynebacterium glutamicum
ggtatcgcagcgctggaggcagtggaggagtggaccactccaaacattgaagcagcattgaacaaggctctcattgaggatctaggcctgaagcctcgcg

SEQ ID NO: 19 - DNA, 100 bp, Corynebacterium glutamicum
caccggctttgaggaactggattcccagtccaaggtcctcggactactttccgatggtgccaagatttctgaagccacagaaggtcaagaagttgaggtc

SEQ ID NO: 20 - DNA, 100 bp, Corynebacterium glutamicum
ggctaaggacctcgttggttcctacgccaaggaactcggtgctgaggctgttatcgtttctgagcacccaggctctgaactggtcggactgacctacgag

SEQ ID NO: 21 - DNA, 100 bp, Corynebacterium glutamicum
tcaccatgaaggtcactcgaccttccgagctcgcaggcgtggattattccgcagaaactgtgatcgctcgtctggaagaggtcggatgcaccgtcgctgt

SEQ ID NO: 22 - DNA, 100 bp, Corynebacterium glutamicum
gtgttcctgattacgccccaccaaaatctgcagcgtttttagcagtccgtgatgcctttgttaatcaagcacataaggccgggtttgagcatattgagct

SEQ ID NO: 23 - DNA, 100 bp, Corynebacterium glutamicum
agtacctggcgcactggaagaatctgacaagaagatccttgatcttgctaccgctgcctttgaatccgttgctgcgaacctggatcagtccaagttcaag

SEQ ID NO: 24 - DNA, 100 bp, Corynebacterium glutamicum
actgttcggccgcgcagaactccgcgacctagacgaaaaaactctcgctgcatccgtttctgaaactgcagttgcagaaatcaaggcaggggagccacgc

SEQ ID NO: 25 - DNA, 100 bp, Corynebacterium glutamicum
atgtctaccgatcacggtagattgttctttatgatcgatctgaaattcctccgtgataacccggacgttgttcgtgcctcccagatcactcgcggcgaag

SEQ ID NO: 26 - DNA, 100 bp, Corynebacterium glutamicum
caccccgcagcagcgtaaggaagctggcatcgatgccaagccaaagcctaaggcggaagcacaggcagaggcacaggctgaagagtctgctgaaaagtaa

SEQ ID NO: 27 - DNA, 100 bp, Corynebacterium glutamicum
acgagtcttggctatcggtgctgagcgcgccaccgaagttgccaacgaaaccttggctgatgtgtacgacaagattggtttcttggcgtctcgtcgctaa

SEQ ID NO: 28 - DNA, 100 bp, Corynebacterium glutamicum
aggtaggcgagtggacgccaggttttgaagttgcgatggatgaggatattgcagttcctaaggctttggctgaaatccataacgctgtccgcgagggcaa

SEQ ID NO: 29 - DNA, 100 bp, Corynebacterium glutamicum
acgtgagcgcacgaaggagcgcaattttcgtgaacaccacagataccgcagtagcaaaccttgttgtttttgaggtccctgcaggcaccgcaattggtgc

SEQ ID NO: 30 - DNA, 100 bp, Corynebacterium glutamicum
atcgaaggcctgccagaagcagtcacctacgaaactcctgtttctgaaaccatcgatgctttggttgattgggctaactccatcgacgtgcagatcgagg

SEQ ID NO: 31 - DNA, 100 bp, Corynebacterium glutamicum
tcgccaccgttttgggtctgcctatcaaggaagttgtcgcaggtggcaacatcgaagaggctgctttcaccgaatctggcgaagcagtcaactctgcgaa

SEQ ID NO: 32 - DNA, 100 bp, Corynebacterium glutamicum
acctggctaacgcacagaaggaattggaaaccaccgcaaagaagctgggtaatgaggctttcctgtccaaggcacctgatgcagtggtagacaagatccg

SEQ ID NO: 33 - DNA, 100 bp, Mycobacterium tuberculosis
cgaggagctcaccggctacaagaagccgatccgggcctgcgcggtagatatcggcgatcggcagtatcgcgagattatttgtggtgcaaccaatttcgcg

SEQ ID NO: 34 - DNA, 100 bp, Mycobacterium tuberculosis
gaggccgggacggtcgcagtgaaggacttgacgacgggtgagcaagtttcggtctcgatggattcggttgtggccgaagtaatttcgcggctggctgggt

SEQ ID NO: 35 - DNA, 100 bp, Mycobacterium tuberculosis
aagcgattgctgaaatggtcgagctcatcgaaaaaatgctggcgtgcggggcggcctatgtcatagaccgggaaatgggagagtaccaggacatctactt

SEQ ID NO: 36 - DNA, 100 bp, Mycobacterium tuberculosis
tcgtggaagtaattgccggcggcaatatttcggaatccgcgtatacaggcgatggcatcctggtcaactcggattacctcaatggaatgagcgtgccagc

SEQ ID NO: 37 - DNA, 100 bp, Pseudomonas aeruginosa
tcctggctgaacgggcagtggatccgcgagcagtccgtggaggagttcgcccgtgaggtgcagaagtgggcgttgaatcccgaatacctgatgaagatcg

SEQ ID NO: 38 - DNA, 100 bp, Pseudomonas aeruginosa
agccctggaactaacggtggtagccgacggggaagggcagtggtcggtcggcgtaccgagccatcgcttcgacatttccctggaagtcgacctgatcgag

SEQ ID NO: 39 - DNA, 100 bp, Pseudomonas aeruginosa
tggttggcgcgccgaccctgcacgactatctcgacgaagaatcgatcgcccacttcgagggtctcaaggcccgcctggatgctgtcggacttcgctacga

SEQ ID NO: 40 - DNA, 100 bp, Pseudomonas aeruginosa
ttccagcccctgatgacccgtatcgagcccgcgaaagtagaagccatgatcgaagcctccaaggaagacctcgccgccgcatcccaacccgccggcaacg

SEQ ID NO: 41 - DNA, 100 bp, Pseudomonas aeruginosa
cgatttcatcaagctgaccttgcaggtctaccgcgacttcggtttcaccgacatcgccatgaagctgtcgacccgtccggccaagcgcgtcggttccgac

SEQ ID NO: 42 - DNA, 100 bp, Pseudomonas aeruginosa
cgaaggcgtcctggaatacaagggccgccgcgacagcgagtcgcagaacctgccgatcggcgaactgatgtcgttcatcaccgaaaaactcagccgctga

SEQ ID NO: 43 - DNA, 100 bp, Pseudomonas aeruginosa
atgcacgagcaatatcagccactcgagatcgaaacccaggcccagaactactggaaagaacaccagtccttcctcgtcagggagctacccgacaaggaga

SEQ ID NO: 44 - DNA, 100 bp, Xylella fastidiosa
ccgagcccagcctgagaagaaagactttatccggcagatcatcagagaagacttagcccacggcacacacactcacatccgcacccgtttccccccagaa

SEQ ID NO: 45 - DNA, 100 bp, Xylella fastidiosa
cgtgaggttggcgcgtttacagtgccagcctactgttaactccatgaacatacctaccaaattcaccacttcaaagattcgtagcgattttctagaattt

SEQ ID NO: 46 - DNA, 100 bp, Xylella fastidiosa
gcagaacttagatcatttggaagttttgcgcgtagcgcttcttggtaaaaatggcagcatcaccttgcagctcaagcaacttggtaaattgccagttgag

SEQ ID NO: 47 - DNA, 100 bp, Xylella fastidiosa
atgctaacgcgcttctcatacaaacggtcagataaaattacgctatctatcgccacacacccacacccacacgtgaaagccccactccgcgccctgattt

SEQ ID NO: 48 - DNA, 100 bp, Xylella fastidiosa
cggtgccgtccacaccgcccctggccacggccaagaagaccaccaagtctttcaacaatacggattactcaatcactacagcgctgccgaactcaacccc

SEQ ID NO: 49 - DNA, 100 bp, Xylella fastidiosa
tggagcgaactggcaatcaccgtgcgcgatgtcattggatcgttacttcgtgacgtgaaactatttgaccgctacgtcggtaagggtatcgaaccaggct

SEQ ID NO: 50 - DNA, 100 bp, Xylella fastidiosa
ttgccacccgttccaccagccatgccgatgcattggtgcgcctggatgcactgatcgaagattcagggccagaggcacacgagatgctgcgccaaggtgt

SEQ ID NO: 51 - DNA, 100 bp, Xylella fastidiosa
tcggtggccacaccatccacccctactcccccttattcacccgcatcgataaaaaagtcattgaagtgatgatcaacgcctccaaagacaccctcgcccc

SEQ ID NO: 52 - DNA, 100 bp, Xylella fastidiosa
taagccacttagtcatgaggatgtattggctaatgcccgtacctacgaggatcaggtatttaaggttttggatcgtacccttaccgaggtgaggttcaat

SEQ ID NO: 53 - DNA, 100 bp, Xylella fastidiosa
gaagaggacatgttcagtactcagctcggtgagcatcggcgctatttgatttcgacctctgaaatttcgctgactaatttagtacgtaacgagattattg

SEQ ID NO: 54 - DNA, 100 bp, Xylella fastidiosa
tgacggtggattcgcgtgccatcgaccagaacaactcagaactgttcaaggttgctagtggcttgagttatgaggatgtactacaagttgaaggcgtcgt

SEQ ID NO: 55 - DNA, 100 bp, Xylella fastidiosa
ggaaggggattcttaatcgtgcgcatgcttataaggcggtggtggataagaatcgtagtgaggggggggatgatgatgccgggatcacggccggtctgtt

SEQ ID NO: 56 - DNA, 100 bp, Xylella fastidiosa
gtgcatgggttggtggcaaagcatcctcccgaagcactacgttacgcgctgctgtcggcacattatcggaaaccgctggactggtcagaggcactcatcg

SEQ ID NO: 57 - DNA, 100 bp, Xylella fastidiosa
agcatgacgccatgctgcgaatcatcactcctgaggatcaagaagctctggacatcatacgtcactcttgtgctcacttggttggccatgcggtcaagca

SEQ ID NO: 58 - DNA, 100 bp, Xylella fastidiosa
actcgttccatctgaacgatgctgatttggtacgtgagtacgaaaatatgagggcgacgtatacacgtatctttaccaggctggggttggagttccgtgc

SEQ ID NO: 59 - DNA, 100 bp, Xylella fastidiosa
aaatacggtttacccatccgtcaagtgatcgcactcaaagaacctaaaaaccaagacgaatccacatgggaaccagatgtctggcgtgattggtacgctg

SEQ ID NO: 60 - DNA, 100 bp, Xylella fastidiosa
ggaagggttgatcgatgtggatgccgagcgcgtgcgcctggataaagaaatcaaacgtgtcgaaagcgaaatcgataaaagcaacgggaaattgagtaac

(19) United States
(12) Patent Application Publication
(10) Pub. No.: US 2007/0134700 A1
(43) Pub. Date: Jun. 14, 2007

(54) Method and array for detection and identification of microorangisms present in a sample using the genomic regions coding for different tRNA synthetases

(75) Inventors: Pablo Andres Moreno Cortez, Lo Barnechea (CL); Andres Octavio Aravena Duarte, Nunoa (CL); Pilar A. Parada Valdecantos, Nunoa (CL)

Correspondence Address: MERCHANT & GOULD PC, P.O. BOX 2903, MINNEAPOLIS, MN, 55402-0903, US

(73) Assignee: BIOSIGMA S.A., Colina (CL)

(21) Appl. No.: 11/601,157

(22) Filed: Nov. 17, 2006

(30) Foreign Application Priority Data
Nov. 21, 2005 (CL) CL 3032-2005

(51) Int. Cl. C12Q 1/68; G06F 19/00

(52) U.S. Cl. 435/6; 702/20

(57) ABSTRACT

The present invention discloses a method to detect and identify microorganisms that are present in a sample, which comprises hybridizing total DNA isolated from the sample with DNA fragments from regions that codifies for tRNA-synthases, hereinafter ‘tRNA-synthases’, said fragments being selected due to their specificity for each taxon to be detected and identified. Furthermore, an array for detection and identification of microorganisms is disclosed, which comprises at least one tRNA-synthase fragment bound to its surface that is specific for each taxon to be detected and identified and has been designed according to the method described in the present document.

### FIELD OF THE INVENTION

The present invention discloses a method to detect and identify microorganisms that are present in a sample, which comprises hybridizing total DNA isolated from the sample, said method using genomic regions that codify for different tRNA-synthases.

### BACKGROUND OF THE INVENTION

Microorganism detection and identification has been carried out using different methods in time. Traditionally, it has been performed using phenotypic, physiological and structural or biochemical features of the microorganisms, by means of methods like microscopy, staining and selective culture, among many others. These techniques allow detecting and identifying a large number of microorganisms suitably, but in many cases they do not differentiate between taxonomically close microorganisms.

In the last years, owing to the advances of molecular biology in the microbiology field, techniques for detection and identification of microorganisms based on the comparison of total or partial sequences of their nucleic acids, either DNA or RNA, has been developed. This makes even feasible the study of species that are not able to be cultured in the laboratory but the DNA of which can be directly extracted from the medium in which they are found. Comparing the conservation and variability features shown by nucleotide sequences of genes that are present in different organisms, it is possible to detect taxonomic groups at different levels, namely kingdom, family, genus or species. It only depends on the amount of available information and the strategy to be followed, understanding by strategy the molecular biology techniques to be employed and the selection of the nucleotide sequences on which work will be carried out.

A widely diffused strategy in the art is working with the gene that codifies for the 16S ribosomal ARN molecule, which will be hereinafter referred as 16S rDNA. This region is characterized by widely variable sequences flanked by highly conserved regions in its sequence. Comparison of variable regions of 16S rDNA has allowed a satisfactory differentiation of different microbiological relevant taxons. On the other hand, the presence of highly conserved regions flanking the variable region has allowed an increase in the sensitivity of identification and detection techniques. These highly conserved regions are common to a large number of different microorganisms, and many times even to entire domains, and this has allowed the use of “universal” primers for Bacteria or Archaea domains, for instance. Using these universal primers in a polymerase chain reaction (PCR), 16S rDNA regions of all microorganisms belonging to said domains that are present in a given metagenomic sample are specifically amplified. Once said 16S rDNA region has been amplified, detection and identification techniques can be applied, based on the recognition of variable regions contained in the amplicon. The use of 16S rDNA being so extended, microorganism classification has been based mainly in the comparison of these sequences until now. The large information existent for 16S rDNA sequences facilitates the design of specific sequences that allow the identification of a determined taxon.

In spite of the virtues of using 16S rDNA, its intra-genus variability has the disadvantage of being low, which makes the differentiation between species of the same genus not always possible. This represents a problem, especially in the biotechnology field, where in many cases it is necessary to work with species having specific activities, the population of which must be monitored and controlled in the processes where they are applied.

When facing this difficulty, it is necessary to have an alternative strategy that allows the detection of taxonomic groups at all levels.

The new proposed genomic region must fulfill the same requirements fulfilled by 16S rDNA, i.e. it must be represented in a large diversity of microorganisms and have at the same time a variability degree in its nucleotide sequence that allow the differentiation of all the relevant taxonomical groups, either families, genera or species. Moreover, it is convenient to consider a collection of sequences belonging to the proposed genomic region to be considered, which has been generated from the largest possible microorganism diversity, as this will assure a better design of specific sequences for each taxonomic level.

We have solved said technical problem by selecting genomic regions codifying for tRNA-synthases to obtain specific sequences for the different microorganism taxons to be detected and identified.

tRNA-synthases are present in all microorganisms and they have been studied and sequenced many times because of their relevance, said sequences being deposited in public databases. This allows the comparison of sequences of genomic regions codifying for tRNA-synthases (herein referred as tRNA-synthases) of many different types of microorganisms, which allows to carry out a good design of specific sequences for detecting and identifying the different relevant taxons.

The selection of the region over which the studies will be carried out is the first stage of a strategy to detect and identify microorganisms. The second stage comprises the molecular biology techniques used in said strategy. There are many molecular biology techniques useful for detecting and identifying microorganisms, which are based in sample DNA hybridization with specific known sequences for a determined taxon. Among them, we can mention fluorescent in situ hybridization (FISH), denaturing gradient gel electrophoresis or temperature gradient gel electrophoresis (DGGE, TGGE), conjugation with specific markers, such as detection probes, polymerase chain reaction (PCR), and macro or micro DNA arrays. All these techniques are able to be performed by using genomic regions codifying for tRNA-synthases as the regions selected for the design of specific sequences for different taxons, and are to be considered within the scope of the invention.

Among all mentioned techniques, we have chosen to work with DNA arrays, because we think it is the most suitable technique for fast microorganism detection and identification, as it allows simultaneous and specific detection of various microorganisms at the same time. Nevertheless, this preference must not be understood as limiting the invention, but as an application example of the same.

A good definition of DNA array is the one proposed by Schena et al. (Trends Biotechnol. 16, 301-306): “a microscopic ordered nucleic acid array that allows simultaneous analysis of complex DNA samples” (Schena M., Heller, R. A., Theriault, P., Konrad, K., Lachenmeier, E. and Davis, R. W. (1998). Depending on the diameter of the deposited DNA spots, there are 2 array types: macro-arrays (300 microns or more) and micro-arrays (less than 100 microns). The first ones can be manually manufactured in the laboratory and the spots can be observed without the help of special equipment. The second ones require an automated deposition process (normally a robotic deposition platform) and a specialized image acquisition and processing equipment.

The selection method using DNA fragment arrays comprise the simultaneous hybridization of the set of array “spots” with a labeled DNA extract of the studied sample. Normally, the DNA from the sample, which has been labeled and fragmented as required, is subjected to a denaturation stage wherein the DNA double helix is separated, e.g. by heating. When temperature is lowered, DNA will tend to couple with its most complementary fragment according to its physicochemical characteristics. Being this DNA in contact with the array, if there is coincidence between sample DNA and the DNA fragment contained in a spot, labeled sample DNA copies will specifically attach to said spot with the largest possibility. This is due to the larger amount of complementary DNA copies contained in the array spot. DNA labeling can be done by any known labeling technique, being fluorescence and radioactive labeling the most common one. Subsequently, in the acquisition and processing stage of the hybridized array image, this label will allow the detection of the microorganisms present in the studied sample.

We have not found in the present state of the art any document that anticipates the subject matter of the present invention. Since now, structural and functional aspects of tRNA-synthases have been studied due to their relevance in protein synthesis (this enzyme promotes the bonding of the different amino acids to their corresponding transference RNA, tRNA) in different microorganisms. Even when genes codifying for tRNA synthases have been sequenced in a large variety of microorganisms, there is no study analyzing their usefulness in identification and classification of microorganisms. There are some studies based on the comparison of tRNA-synthase proteins that have been sequenced from different microorganisms, in order to establish evolutionary relationships between these genes and the phylogeny of bacteria containing them (as, for example, the review of O'Donoghue P. (MMBR 2003 67 (4):550-573)), where their sequences and structures are compared. Nevertheless, there is no suggestion about the analysis of tRNA-synthase genes for taxonomic classification, and not even about its systematic use in molecular biology techniques for microorganism detection and identification, as we propose in the present invention.

### BRIEF DESCRIPTION OF THE INVENTION

The present invention discloses a method and array to detect and identify microorganisms that are present in a sample by using genomic regions codifying for different tRNA synthases.

Due to their relevance, tRNA-synthases are represented in substantially all known microorganisms. Moreover, the genes that codify for them are sufficiently variable in their nucleotide sequence as to allow distinguishing between different microorganism taxons. That is, it is possible to find specific sequences for a determined genus or species.

The present invention discloses a method to detect and identify microorganisms that are present in a sample, comprising hybridizing total DNA isolated from the sample with DNA regions codifying for tRNA synthases selected for their specificity for each type of microorganism to be identified. The method involves designing the specific DNA regions for each relevant taxon, from a database that comprises the sequences of different described tRNA-synthases.

Once specific sequences for each relevant microorganism are obtained, these can be applied in diverse molecular biology detection techniques, such as PCR, FISH, DGGE, TGGE, detection probes, DNA arrays, or any other technique based in specific hybridization of nucleic acids.

As an example of the invention, DNA arrays having specific sequences of tRNA-synthases for the detection and identification of Agrobacterium tumefaciens; Corynebacterium glutamicum; Mycobacterium tuberculosis; Pseudomonas aeruginosa and Xylella fastidiosa have been developed.

### BRIEF DESCRIPTION OF THE FIGURES

FIG. 1 shows the result of the hybridization of a metagenomic sample (M1) obtained from a mixture of commercial strains of Agrobacterium tumefaciens, Corynebacterium glutamicum and Mycobacterium tuberculosis, using an array of the invention. Used tRNA-synthase fragments are described in Table 3. Each fragment was deposited by triplicate. In Table 1, the content of each position in microarray 1 is detailed.

Conclusion: The results verify the presence of Agrobacterium tumefaciens, Corynebacterium glutamicum and Mycobacterium tuberculosis in M1. All positive controls showed a hybridization signal and negative controls remained unmarked.

FIG. 2 shows the result of the hybridization of a second metagenomic sample (M2) obtained from a mixture of commercial strains of Pseudomonas aeruginosa and Xylella fastidiosa, using another array of the invention. Used tRNA-synthase fragments are described in Table 3. Each fragment was deposited by triplicate. In Table 2, the content of each position in microarray 2 is detailed.

Conclusion: The results verify the presence of Pseudomonas aeruginosa and Xylella fastidiosa in M2. All positive controls showed a hybridization signal and negative controls remained unmarked.

### DETAILED DESCRIPTION OF THE INVENTION

tRNA-synthases are essential components of the translation machinery in protein synthesis within cells, both in prokaryotes and in eukaryotes. These enzymes are responsible for binding each tRNA with its corresponding amino acid, catalyzing a specific amino-acylation reaction. Owing to reaction specificity, there is a specific tRNA-synthase for each tRNA-amino acid pair, and therefore each cell has at least 20 different tRNA-synthases, one for each of the natural 20 amino acids. Obviously, each of these 20 tRNA-synthases is codified in the genome, representing at least 20 different genomic regions in all organisms.

As has been mentioned before, the present invention relates to the use of said genomic regions codifying for the different tRNA-synthases in molecular biology techniques for microorganism detection and identification.

For a genomic region to be useful as template in molecular biology techniques for detection and identification of various microorganisms, it must fulfill 2 basic requirements: first, it has to be present in all microorganisms to be detected or identified. This requirement is fully fulfilled by tRNA-synthases, as they are not only present in all microorganisms, but also in all known organisms. The second requirement is that sequences have to be sufficiently variable to have significant differences between different taxons to be studied; this requirement is also fulfilled by tRNA-synthases as a group.

A third non-essential requirement that facilitates working with a particular genomic region is the existence of a great deal of information available for said region. This requirement is not essential, as it is always possible to generate the required information, but this considerably increases the operation cost. In this case, due to the relevance of tRNA-synthases, many researchers have sequenced them for different microorganisms, public information being available for more than 200 different microorganisms.

If tRNA-synthase sequences are to be used in molecular biology techniques for microorganism detection and identification, a database containing tRNA-synthase sequences from taxonomically classified microorganisms must be available in the first place. An example of a database that can be used is GenBank, from NCBI (Benson D A, Boguski M S, Lipman D J, and Ostell J. (1997) GenBank. Nucleic Acids Res. January 1; 25(1):1-6). We have processed the information on tRNA-synthases available in said public database to work more efficiently, thus creating our own database.

Once the database to be used has been selected, DNA fragments must be designed that are taxon specific for each relevant microorganism, having a size suitable for the molecular biology technique to be used. For example, if PCR is to be used, a pair of primers having 15 to 30 nucleotides must be designed, said pair of primers being separated by a distance of 200 to 3,000 nucleotides. If a microarray is to be used, a specific fragment having 50 to 70 nucleotides, in case of using synthetic oligonucleotides, or specific fragments having 200 to 3,000 nucleotides, if PCR products are to be deposited, must be designed, and likewise for each available molecular biology technique.

The design of these taxon-specific tRNA-synthase fragments can be carried out by using any method available in the art, without limiting the scope of the present invention.

In our particular case, the design of DNA fragments of tRNA-synthases was carried out using a proprietary method protected by patent application CL 2102-05, owned by Biosigma. Said method is applied in the oligonucleotide design software “Massive Primer Designer”. For more clarity, we will briefly explain the method.

A database containing all tRNA-synthase sequences for microorganisms disclosed up to date in the literature, corresponding to 230 microorganisms, was used. In a first stage, we will refer to nucleotide sequences as words having defined length in the alphabet {A,C,T,G}. Each sequence is computationally scanned from 5′ to 3′ obtaining all existing words for the desired DNA fragments or oligonucleotides to be designed. Each found word is then considered an oligonucleotide candidate. This oligonucleotide candidate pass through the following tests in the same detailed order, wherein a rejection in one of the tests means the total elimination of the candidate.
1. GC composition level: This is a filter that allows discarding a priori candidates that have very high or very low values of hybridization temperature, by performing a very cheap calculus in terms of time. The candidate is rejected if its GC composition falls off the limits imposed at the moment of executing the software.
2. Hybridization temperature: Given a sequence and environmental conditions (salt concentration, nucleotide concentration, etc.), the hybridization temperature between said sequence and its complementary one is calculated. When executing the software, an oligonucleotide is rejected if its hybridization temperature falls off the established limits to be used during a hybridization assay.
3. Secondary structure: for a reference temperature, which depends on the particular molecular biology technique to be used, it is assessed whether the candidate sequence stabilizes itself by forming a stable secondary structure (three-dimensional fold of the oligonucleotide alone) or not. If a secondary structure is formed, the oligonucleotide is replaced by its reverse complementary sequence; if this reverse complementary also forms a secondary structure, then the oligonucleotide is rejected. If only the original form of the oligonucleotide forms a secondary structure, but not its reverse complementary sequence, then this latter is selected as candidate oligonucleotide for the following tests.
4. Specificity: using one or more sequence alignment algorithms, the candidate is tested for a relevant similarity with sequences pertaining to the other microorganisms.

In this method some other optimizations are included, such as the initial selection of sequences that are relatively specific for each microorganism, in order to limit the oligonucleotide search in selected regions.

By using this method, DNA fragments can be obtained from regions codifying for tRNA-synthases that fulfill all the described requirements and can be employed in a molecular biology technique for the detection and identification of any relevant microorganism.

The database used by us contains tRNA-synthase sequences disclosed in the literature for microorganisms belonging to the following taxons:

Acinetobacter spp.; Aeropyrum pernix; Agrobacterium tumefaciens; Anabaena spp.; Anaplasma marginale; Aquifex aeolicus; Archaeoglobus fulgidus; Azoarcus spp.; Bacillus anthracis; Bacillus cereus; Bacillus clausii; Bacillus halodurans; Bacillus licheniformis; Bacillus subtilis; Bacillus thuringiensis; Bacteroides fragilis; Bacteroides thetaiotaomicron; Bartonella henselae; Bartonella quintana; Bdellovibrio bacteriovorus; Bifidobacterium longum; Blochmannia floridanus; Bordetella bronchiseptica; Bordetella parapertussis; Bordetella pertussis; Borrelia burgdorferi; Borrelia garinii; Bradyrhizobium japonicum; Brucella abortus; Brucella melitensis; Brucella suis; Buchnera aphidicola; Burkholderia mallei; Burkholderia pseudomallei; Campylobacter jejuni; Caulobacter crescentus; Chlamydia muridarum (Chlamydia trachomatis MoPn); Chlamydia trachomatis; Chlamydophila abortus; Chlamydophila caviae; Chlamydophila pneumoniae; Chlorobium tepidum; Chromobacterium violaceum; Clostridium acetobutylicum; Clostridium perfringens; Clostridium tetani; Corynebacterium diphtheriae gravis; Corynebacterium efficiens; Corynebacterium glutamicum; Coxiella burnetii; Dehalococcoides ethenogenes; Deinococcus radiodurans; Desulfotalea psychrophila; Desulfovibrio vulgaris; Ehrlichia ruminantium; Enterococcus faecalis; Erwinia carotovora atroseptica; Escherichia coli; Francisella tularensis; Fusobacterium nucleatum; Geobacillus kaustophilus; Geobacter sulfurreducens; Gloeobacter violaceus; Gluconobacter oxydans; Haemophilus ducreyi; Haemophilus influenzae; Haloarcula marismortui; Halobacterium sp.; Helicobacter hepaticus; Helicobacter pylori; Idiomarina loihiensis; Lactobacillus acidophilus; Lactobacillus johnsonii; Lactobacillus plantarum; Lactococcus lactis subsp. lactis; Legionella pneumophila; Leifsonia xyli xyli; Leptospira interrogans; Leptospira interrogans; Listeria innocua; Listeria monocytogenes; Listeria monocytogenes; Mannheimia succiniciproducens; Mesoplasma florum; Mesorhizobium loti; Methanobacterium thermoautotrophicum; Methanococcus jannaschii; Methanococcus maripaludis; Methanopyrus kandleri; Methanosarcina acetivorans; Methanosarcina mazei; Methylococcus capsulatus; Mycobacterium avium paratuberculosis; Mycobacterium bovis bovis; Mycobacterium leprae; Mycobacterium tuberculosis; Mycoplasma gallisepticum; Mycoplasma genitalium; Mycoplasma hyopneumoniae; Mycoplasma mobile; Mycoplasma mycoides mycoides; Mycoplasma penetrans; Mycoplasma pneumoniae; Mycoplasma pulmonis; Nanoarchaeum equitans; Neisseria gonorrhoeae; Neisseria meningitidis; Nitrosomonas europaea; Nocardia farcinica; Oceanobacillus iheyensis; Parachlamydia sp.; Pasteurella multocida; Photobacterium profundum; Photorhabdus luminescens laumondii; Phytoplasma spp.; Picrophilus torridus; Porphyromonas gingivalis; Prochlorococcus marinus; Propionibacterium acnes; Pseudomonas aeruginosa; Pseudomonas putida; Pseudomonas syringae; Pyrobaculum aerophilum; Pyrococcus abyssi; Pyrococcus furiosus; Pyrococcus horikoshii; Ralstonia solanacearum; Rhodopirellula baltica; Rhodopseudomonas palustris; Rickettsia conorii; Rickettsia prowazekii; Rickettsia typhi; Salmonella enterica; Salmonella typhi; Salmonella typhimurium; Shewanella oneidensis; Shigella flexneri; Silicibacter pomeroyi; Sinorhizobium meliloti; Staphylococcus aureus; Staphylococcus epidermidis; Streptococcus agalactiae; Streptococcus mutans; Streptococcus pneumoniae; Streptococcus pyogenes; Streptococcus thermophilus; Streptomyces avermitilis; Streptomyces coelicolor; Sulfolobus solfataricus; Sulfolobus tokodaii; Symbiobacterium thermophilum; Synechococcus spp.; Synechocystis spp.; Thermoanaerobacter tengcongensis; Thermococcus kodakaraensis; Thermoplasma acidophilum; Thermoplasma volcanium; Thermosynechococcus elongatus; Thermotoga maritima; Thermus thermophilus; Treponema denticola; Treponema pallidum; Tropheryma whipplei; Ureaplasma urealyticum; Vibrio cholerae; Vibrio fischeri; Vibrio parahaemolyticus; Vibrio vulnificus; Vibrio vulnificus; Wigglesworthia brevipalpis; Wolbachia spp.; Wolinella succinogenes; Xanthomonas axonopodis; Xanthomonas campestris; Xanthomonas oryzae; Xylella fastidiosa; Yersinia pestis; Yersinia pestis; Yersinia pseudotuberculosis and Zymomonas mobilis.

In a preferred embodiment of the present invention, DNA arrays are developed with the designed tRNA-synthase fragments. In the examples, we have included 60 tRNA-synthase sequences specific for 5 microorganisms: Agrobacterium tumefaciens; Corynebacterium glutamicum; Mycobacterium tuberculosis; Pseudomonas aeruginosa and Xylella fastidiosa. Specific arrays containing these fragments are especially protected.

It should be noted that the arrays contained in the present invention are those comprising at least one of the DNA fragments included in sequences N^{o} 1 to 60, either entirely, or in a larger region comprising it, such as a PCR product, or partially as one of the sub-fragments contained in each of the fragments herein disclosed, or as the reverse complementary sequences of any of the former options. This is vitally relevant, as the specificity of a nucleotide sequence is the same specificity of its reverse complementary sequence, and it is this feature, i.e. specificity, the more difficult goal to achieve in the design of the DNA fragments to be used in an array. It could be possible that the stability of the reverse complementary sequence should not be suitable for the sequence to be used in an array, but nevertheless the skilled person will distinguish between thermodynamically stable and unstable oligonucleotides by means of diverse tools existing in the art. All reverse complementary sequences of fragments N^{o}1 to 60 of the present invention, either entirely, or in a larger region comprising it, such as a PCR product, or partially as one of the sub-fragments contained in each of the fragments herein disclosed, are to be considered within the scope of the present invention.

The efficiency of the arrays of the invention is given by the specificity and stability of the fragments to be deposited. These characteristics are retained by each sub-fragment contained within the designed fragments. This means that specificity is retained if nucleotide 1 to 100 or 42 to 92, or 15 to 65, or any other possible selection is used. All selections are sub-fragments and are comprised within the scope of the present invention.

It is also possible to have DNA fragments that contain fragments or sub-fragments of the invention flanked by other oligonucleotides, either by synthesis or as PCR products. These larger fragments that contain the fragments of the present disclosure, the specificity of which is given by the fragments or sub-fragments designed by us, are also to be considered within the scope of the present invention.

Each selected fragment or sub-fragment have to be synthesized in many hundreds of copies and deposited as a homogeneous spot on a suitable support for an array, such as glass, silicone, nylon or other support in the art. Synthesis techniques for DNA fragments and array manufacture are known, and any of them could be used to manufacture the arrays of the present invention.

### EXAMPLES

### Example 1

### Design of Specific Sequences for 5 Different Microorganisms

A total of 60 fragments of tRNA-synthases having 100 nucleotides that are specific for species Agrobacterium tumefaciens; Corynebacterium glutamicum; Mycobacterium tuberculosis; Pseudomonas aeruginosa or Xylella fastidiosa were designed. The sequences of all 60 designed tRNA-synthase fragments were included in the list of sequences.

To design these specific fragments, a database comprising tRNA-synthase sequences for 230 microorganisms selected from NCBI's GenBank public database was first constructed.

In each case, a set of 100-letter oligonucleotides present in each sequence was determined, discarding those appearing more than once in each sequence, considering up to 3 substitutions. These oligonucleotides were the “candidate oligonucleotides”, which were evaluated according to their thermodynamic stability. Subsequently, thermodynamically favorable fragments were aligned against the entire database, in order to determine their specificity. Best fragments were selected from different tRNA-synthases for detection and identification of each of the species Agrobacterium tumefaciens; Corynebacterium glutamicum; Mycobacterium tuberculosis; Pseudomonas aeruginosa or Xylella fastidiosa.

Of all 60 designed sequences, sequences N^{o} 1 to 17 are specific for Agrobacterium tumefaciens; sequences N^{o}18 to 32 are specific for Corynebacterium glutamicum; sequences N^{o}33 to 36 are specific for Mycobacterium tuberculosis; sequences N^{o}37 to 43 are specific for Pseudomonas aeruginosa; and sequences N^{o}44 to 60 are specific for Xylella fastidiosa.

The designed fragments can be deposited on the array either entire, or comprised in a larger fragment that contains them, or in partial form as any of the sub-fragments comprised in the fragment, or as the reverse complementary sequences of any of the former options. Advantageously, sub-fragments having 50 or 70 nucleotides were deposited, preferably sub-fragments having 60 nucleotides.

### Example 2

### Microarrays to Detect and Identify the Presence of Microorganisms

Two microarrays were manufactured with the tRNA-synthase fragments designed in the former example, which specifically identify: Agrobacterium tumefaciens; Corynebacterium glutamicum; Mycobacterium tuberculosis; Pseudomonas aeruginosa or Xylella fastidiosa.

In each microarray, one 60-nucleotide sub-fragment for each of the designed fragments for each species to be detected, one positive control and three negative controls were included.

In the first microarray, specific sub-fragments for Agrobacterium tumefaciens; Corynebacterium glutamicum and Mycobacterium tuberculosis were deposited. In the following Table 1, the content of each position in microarray 1 is detailed.

TABLE 1

| | Position in microarray 1 |
|---|---|
| Microorganism | tRNA-synthase |
| Agrobacterium tumefaciens | A1-A3 |
| Corynebacterium glutamicum | B4-B6 |
| Mycobacterium tuberculosis | C4-C6 |
| Positive control | B1-B3 |
| Negative control | C1-C3; A4-A6; A7-A9 |

Each fragment was deposited by triplicate. The manufacture of the microarray was performed by a specialized company in the field.

In the second microarray, specific sub-fragments for Pseudomonas aeruginosa and Xylella fastidiosa were deposited. In the following Table 2, the content of each position in microarray 2 is detailed.

TABLE 2

| | Position in microarray 2 |
|---|---|
| Microorganism | tRNA-synthase |
| Pseudomonas aeruginosa | C1-C3 |
| Xylella fastidiosa | A4-A6 |
| Positive control | B4-B6 |
| Negative control | A1-A3; B1-B3; C4-C6 |

Each fragment was deposited by triplicate. The manufacture of the microarray was performed by a specialized company in the field.

The 60-nucleotide sub-fragments deposited on the microarrays were selected from the fragments displayed in Table 3, which are defined in the list of sequences.

TABLE 3

tRNA-synthase

| Microorganism | Amino-acyl | Sequence N ° |
|---|---|---|
| Agrobacterium tumefaciens | Alanyl | 2 |
| Corynebacterium glutamicum | Histidyl | 22 |
| Mycobacterium tuberculosis | Phenylalanyl | 33 |
| Pseudomonas aeruginosa | Methionyl | 40 |
| Xylella fastidiosa | Arginyl | 47 |

### Example 3

### Use of the Obtained Microarrays to Detect and Identify Microorganisms

The microarrays obtained in Example 2 were used with metagenomic samples obtained from a mixture of commercial strains of each of the species contained in the microarray. Two samples were analyzed, one hybridizing on each microarray, the first one containing a mixture of Agrobacterium tumefaciens, Corynebacterium glutamicum and Mycobacterium tuberculosis (M1), and the second one comprising a mixture of Pseudomonas aeruginosa and Xylella fastidiosa (M2).

Total DNA was extracted from M1 and M2 using traditional DNA extraction methods.

2 μl were taken from the DNA samples, which had a concentration between 1 and 5 μg/μl, and were put in Eppendorf tubes. In each case, the following method was carried out:

36 μl of ddH₂O and 3.3 ml of 6-nucleotide random primers were added. The mix was boiled for 5 minutes and then the assay was continued on ice.

2 μl of a nucleotide mix were added, where dUTP was labeled with Cy fluorophore. Cy5 was used for M1, with green fluorescence, while Cy3 was used for M2, with red fluorescence. Subsequently, 4 μl of a polymerase and 5 μl of buffer solution were added, and the mix was incubated for 4 hours at 37° C.

The reaction was stopped with 5 μl 0.5 M EDTA, pH 8. Labeled DNA was recovered using an ion exchange column. The eluate containing DNA was dried under vacuum.

DNA was resuspended by adding 100 μl of a buffer solution. Resuspended samples were mixed with the complementary oligonucleotide of the positive control deposited on the microarray, which in each case was labeled with the inverted Cy fluorophore of the problem sample. In this way, the positive control of sample 1 will have red fluorescence and the positive control of sample 2 will have green fluorescence.

The samples thus prepared were subjected to 100° C. for one and a half minute to denature DNA. M1 was hybridized overnight on microarray 1 and M2 was hybridized overnight on microarray 2, both at 55° C.

The following morning, each microarray was washed twice with 2×SSC, 0.1% SDS, at 45° C.; once with 0.2×SSC, 0.1% SDS, at 42° C., and once with 0.2×SSC, at 42° C.

Each microarray was put in a case with MilliQ water for 15 minutes and subsequently dried by centrifugation in a Falcon tube for 1 minute at 1100 rpm.

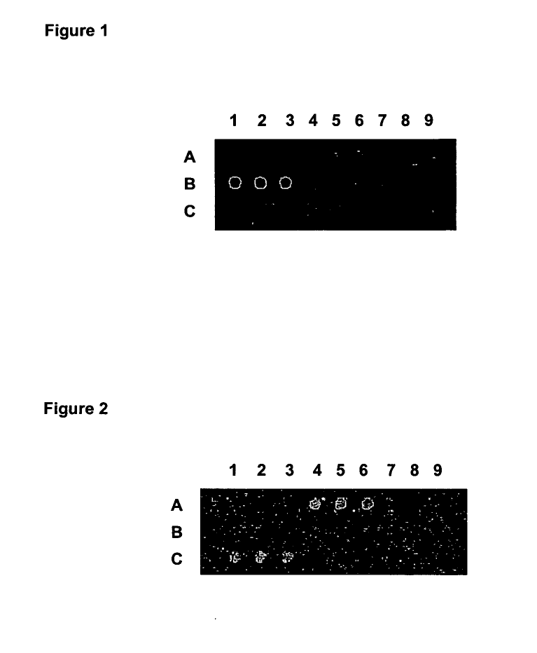

Finally, the results of the microarrays could be observed, which are shown in FIG. 1 for M1 and in FIG. 2 for M2.

In Table 4, the positions of the different fragments in microarray 1 are indicated and the result of hybridizations with DNA obtained from M1, which is shown in FIG. 1, is summarized. It is possible to observe that in all cases the families of spots (replicates of the same tRNA-synthase fragment) had always the same result. All positive controls showed hybridization and negative controls remained unmarked.

TABLE 4
Sample 1 (M1)

Position in the microarray

| Microorganism | tRNA-synthase 1 | R |
|---|---|---|
| Agrobacterium tumefaciens | A1-A3 | + |
| Corynebacterium glutamicum | B4-B6 | + |
| Mycobacterium tuberculosis | C4-C6 | + |
| Positive control | B1-B3 | + |
| Negative control | C1-C3; A4-A6; A7-A9 | − |

Legend:
(R): result;
(+): positive;
(−): negative.

The results verify the presence of Agrobacterium tumefaciens, Corynebacterium glutamicum and Mycobacterium tuberculosis in M1.

In Table 5, the positions of the different fragments in microarray 2 are indicated and the result of hybridizations with DNA obtained from M2, which is shown in FIG. 2, is summarized. Like for M1, it is possible to observe that the families of spots had always the same result. All positive controls showed hybridization and negative controls remained unmarked.

TABLE 5
Sample 2 (M2)

Position in the microarray

| Microorganism | tRNA-synthase 1 | R |
|---|---|---|
| Pseudomonas aeruginosa | C1-C3 | + |
| Xylella fastidiosa | A4-A6 | + |
| Positive control | B4-B6 | + |
| Negative control | A1-A3; B1-B3; C4-C6 | − |

Legend:
(R): result;
(+): positive;
(−): negative.

The results verify the presence of Pseudomonas aeruginosa and Xylella fastidiosa in M2.

List of Sequences

Sequence No.: 1

Length: 100

Type: DNA

Microorganism: Agrobacterium tumefaciens

Category: Glutamyl and Glutaminyl tRNA-synthase

Sequence:

CGG TTT TTC GCG CAC TTG CCT TGT CCC GAG GGG ATG
AGG CAT AGT CCG CGA ATC AAA AAC GGC CTG TTC CGT
TTC GGG GAA GCG CAC GAT TCC GTT GCCG

Sequence No.: 2

Length: 100

Type: DNA

Microorganism: Agrobacterium tumefaciens

Category: Alanyl tRNA-synthase

Sequence:

GGT CTT GCC GAT GAG GCC AAG GCC AAT CTC GGC TCC
GGC GTT GTG TTG CTG ATT GCG GTT TCT GAC GAT GGC
AAG GCA AGC GCC GTC GCT GCC GTG ACCG

Sequence No.: 3

Length: 100

Type: DNA

Microorganism: Agrobacterium tumefaciens

Category: Phenylalanyl tRNA-synthase alpha subunit

Sequence:

ATG ACT GAA CTT GAT ACC TTG AAA TCG CAA CTG ATG
TCG GAG ATC GCC GCC GCT GCC GAT GAG CCG GCC ATC
GAG GCC GTG CGC GTC TCC GCG CTC GGCA

Sequence No.: 4

Length: 100

Type: DNA

Microorganism: Agrobacterium tumefaciens

Category: Arginyl tRNA-synthase

Sequence:

ACG CGC TCG AGA CCC TCG ATC TCG TGA AAG AGA ACC
GCG AAA AGG TCG ATT TCA GTC GAA TTA CCG TCG AAT
CCC CGC GTG ATC TCA GCC ATG GCG ATGT

Sequence No.: 5

Length: 100

Type: DNA

Microorganism: Agrobacterium tumefaciens

Category: Isoleucyl tRNA-synthase

Sequence:

CAA GCC ATG GCC GCG AGG ACT TTG ATG CCT GGA CGA
ATA GCG TCC GCA CCC TTG AAG CAC GCG GGA TCG ATA
CCA AAA TCC CGT TCC CGG TCG ATG ACGC

Sequence No.: 6

Length: 100

Type: DNA

Microorganism: Agrobacterium tumefaciens

Category: Phenylalanyl tRNA-synthase beta subunit

Sequence:

AAC TTC GAG ACC GGT CAG GCG CTT CAC TTC CGA CAG
CGG AAA ATC CAC GAC CTT GGG CTG ATG CCC CTT GTA
ACC GAC GAT CTT GGC TTC GCC AGC GATG

Sequence No.: 7

Length: 100

Type: DNA

Microorganism: Agrobacterium tumefaciens

Category: Histidyl tRNA-synthase

Sequence:

AGG GTG CGG GTC TTG GCG ATG AAC AGA TCG AAA AAG
TGC TGT TCT TCG TTG GTA TCA AGG ATT ATG CGG CAA
GCG CTG ATG ATC TCG CAA AAC TGG TTGC

Sequence No.: 8

Length: 100

Type: DNA

Microorganism: Agrobacterium tumefaciens

Category: Methionyl tRNA-synthase

Sequence:

AGA CCT CGC GCA ATA TCT GGA ACC TGA TGG CCG ATA
GCG GCG ACA TCT ACA AGG ACA GCT ATG CCG GCT GGT
ATT CCG TGC GCG ACG AGG CCT ATT ACGC

Sequence No.: 9

Length: 100

Type: DNA

Microorganism: Agrobacterium tumefaciens

Category: Tyrosyl tRNA-synthase

Sequence:

TTC AAG GAG GAG GCC CGC AAG CTA ATG ACG ATC GAC
ATG ATC GAG GAC AAT ATC ACC TCG CTC AAG CAC GTC
TTT GCG AAT TAC CTC GAT TAC GAT CGTG

Sequence No.: 10

Length: 100

Type: DNA

Microorganism: Agrobacterium tumefaciens

Category: Seryl tRNA-synthase

Sequence:

ATG GCG CAG AAG AAC ATG GAA CTG GCG GAA AAG CTG
AAA GCC GAA GTC GCT GAT ATC AAG GAA AAC ATG CCG
CGC GCC GAG GAG GAG GAC CGT CAG GTCT

Sequence No.: 11

Length: 100

Type: DNA

Microorganism: Agrobacterium tumefaciens

Category: Aspartyl tRNA-synthase

Sequence:

CAT CCG CAT CGA CGG TCT AGT CAA GGC GCG TTC GGA
AGA CAC CAT CAA CAA AGG CAT GGC GAC CGG CGA GAT
CGA GCT TTA CGC GCA GGA GAT CGA AGTT

Sequence No.: 12

Length: 100

Type: DNA

Microorganism: Agrobacterium tumefaciens

Category: Tryptophanyl tRNA-synthase

Sequence:

ACA TCG ATT TCG GCG GCC ATA TTC GCA ACG CCG GTC
TGG GCG TTA ATA TCA CGG TTG GCG ATG AGC CGG TGC
ACG CTT ATT TCC CGA TGG TGG AGC CGCT

Sequence No.: 13

Length: 100

Type: DNA

Microorganism: Agrobacterium tumefaciens

Category: Cysteinyl tRNA-synthase

Sequence:

CCG GTT GCG GGA TGA GCT TTC CGA GAA GGG TAT TCA
GCT GAA GGA TGG CAA GGA CAA GGA GAC TGG GGA ACG
GAC GAC GAC GTG GGA GTT GAA GCG GTAG

Sequence No.: 14

Length: 100

Type: DNA

Microorganism: Agrobacterium tumefaciens

Category: Threonyl tRNA-synthase

Sequence:

ACC GTC AAC ATT CGC CGT CTC GGT TCG CAG AAC CAG
ACG TCG ATG TCG CTC GAA GAG GCG ATC ACC AGC CTC
GTG GAC GAG GCG ACG CCG CCG GAT GTCA

Sequence No.: 15

Length: 100

Type: DNA

Microorganism: Agrobacterium tumefaciens

Category: Prolyl tRNA-synthase

Sequence:

CGC TCC TTT CGA TGT CGT GAT CAT CAA CAT GAA GGC
TGG CGA TGC GGC CTG CGA TGC GGC TTG TGA AAA GCT
GTA TTA TCA GCT CTC CAA CGC CGG CAAG

Sequence No.: 16

Length: 100

Type: DNA

Microorganism: Agrobacterium tumefaciens

Category: Leucyl tRNA-synthase

Sequence:

CCG AAG CCG GCG ACC GAA GGC GAA GGG CTG GCG GCC
TCC AAG GCA GCC CAC AAG ACG CTG AAG GCT GTT CAG
GAA GAT CTG GAC AAG CTC GCC TTC AACA

Sequence No.: 17

Length: 100

Type: DNA

Microorganism: Agrobacterium tumefaciens

Category: Valyl tRNA-synthase

Sequence:

GAC TTC AAC GAT TTC GAA GTC GGC AAG CGT TGC GGC
CTG CGC GCC ATC AAC GTC ATG AAC ATC GAT GGT ACG
ATC TCC ATC AAG GAA AAC GAA GAT TTCC

Sequence No.: 18

Length: 100

Type: DNA

Microorganism: Corynebacterium glutamicum

Category: Glutamyl and Glutaminyl tRNA-synthase

Sequence:

GGT ATC GCA GCG CTG GAG GCA GTG GAG GAG TGG ACC
ACT CCA AAC ATT GAA GCA GCA TTG AAC AAG GCT CTC
ATT GAG GAT CTA GGC CTG AAG CCT CGCG

Sequence No.: 19

Length: 100

Type: DNA

Microorganism: Corynebacterium glutamicum

Category: Alanyl tRNA-synthase

Sequence:

CAC CGG CTT TGA GGA ACT GGA TTC CCA GTC CAA GGT
CCT CGG ACT ACT TTC CGA TGG TGC CAA GAT TTC TGA
AGC CAC AGA AGG TCA AGA AGT TGA GGTC

Sequence No.: 20

Length: 100

Type: DNA

Microorganism: Corynebacterium glutamicum

Category: Isoleucyl tRNA-synthase

Sequence:

GGC TAA GGA CCT CGT TGG TTC CTA CGC CAA GGA ACT
CGG TGC TGA GGC TGT TAT CGT TTC TGA GCA CCC AGG
CTC TGA ACT GGT CGG ACT GAC CTA CGAG

Sequence No.: 21

Length: 100

Type: DNA

Microorganism: Corynebacterium glutamicum

Category: Phenylalanyl tRNA-synthase beta subunit

Sequence:

TCA CCA TGA AGG TCA CTC GAC CTT CCG AGC TCG CAG
GCG TGG ATT ATT CCG CAG AAA CTG TGA TCG CTC GTC
TGG AAG AGG TCG GAT GCA CCG TCG CTGT

Sequence No.: 22

Length: 100

Type: DNA

Microorganism: Corynebacterium glutamicum

Category: Histidyl tRNA-synthase

Sequence:

GTG TTC CTG ATT ACG CCC CAC CAA AAT CTG CAG CGT
TTT TAG CAG TCC GTG ATG CCT TTG TTA ATC AAG CAC
ATA AGG CCG GGT TTG AGC ATA TTG AGCT

Sequence No.: 23

Length: 100

Type: DNA

Microorganism: Corynebacterium glutamicum

Category: Methionyl tRNA-synthase

Sequence:

AGT ACC TGG CGC ACT GGA AGA ATC TGA CAA GAA GAT
CCT TGA TCT TGC TAC CGC TGC CTT TGA ATC CGT TGC
TGC GAA CCT GGA TCA GTC CAA GTT CAAG

Sequence No.: 24

Length: 100

Type: DNA

Microorganism: Corynebacterium glutamicum

Category: Tyrosyl tRNA-synthase

Sequence:

ACT GTT CGG CCG CGC AGA ACT CCG CGA CCT AGA CGA
AAA AAC TCT CGC TGC ATC CGT TTC TGA AAC TGC AGT
TGC AGA AAT CAA GGC AGG GGA GCC ACGC

Sequence No.: 25

Length: 100

Type: DNA

Microorganism: Corynebacterium glutamicum

Category: Seryl tRNA-synthase

Sequence:

ATG TCT ACC GAT CAC GGT AGA TTG TTC TTT ATG ATC
GAT CTG AAA TTC CTC CGT GAT AAC CCG GAC GTT GTT
CGT GCC TCC CAG ATC ACT CGC GGC GAAG

Sequence No.: 26

Length: 100

Type: DNA

Microorganism: Corynebacterium glutamicum

Category: Aspartyl tRNA-synthase

Sequence:

CAC CCC GCA GCA GCG TAA GGA AGC TGG CAT CGA TGC
CAA GCC AAA GCC TAA GGC GGA AGC ACA GGC AGA GGC
ACA GGC TGA AGA GTC TGC TGA AAA GTAA

Sequence No.: 27

Length: 100

Type: DNA

Microorganism: Corynebacterium glutamicum

Category: Tryptophanyl tRNA-synthase

Sequence:

ACG AGT CTT GGC TAT CGG TGC TGA GCG CGC CAC CGA
AGT TGC CAA CGA AAC CTT GGC TGA TGT GTA CGA CAA
GAT TGG TTT CTT GGC GTC TCG TCG CTAA

Sequence No.: 28

Length: 100

Type: DNA

Microorganism: Corynebacterium glutamicum

Category: Cysteinyl tRNA-synthase

Sequence:

AGG TAG GCG AGT GGA CGC CAG GTT TTG AAG TTG CGA
TGG ATG AGG ATA TTG CAG TTC CTA AGG CTT TGG CTG
AAA TCC ATA ACG CTG TCC GCG AGG GCAA

Sequence No.: 29

Length: 100

Type: DNA

Microorganism: Corynebacterium glutamicum

Category: Threonyl tRNA-synthase

Sequence:

ACG TGA GCG CAC GAA GGA GCG CAA TTT TCG TGA ACA
CCA CAG ATA CCG CAG TAG CAA ACC TTG TTG TTT TTG
AGG TCC CTG CAG GCA CCG CAA TTG GTGC

Sequence No.: 30

Length: 100

Type: DNA

Microorganism: Corynebacterium glutamicum

Category: Prolyl tRNA-synthase

Sequence:

ATC GAA GGC CTG CCA GAA GCA GTC ACC TAC GAA ACT
CCT GTT TCT GAA ACC ATC GAT GCT TTG GTT GAT TGG
GCT AAC TCC ATC GAC GTG CAG ATC GAGG

Sequence No.: 31

Length: 100

Type: DNA

Microorganism: Corynebacterium glutamicum

Category: Leucyl tRNA-synthase

Sequence:

TCG CCA CCG TTT TGG GTC TGC CTA TCA AGG AAG TTG
TCG CAG GTG GCA ACA TCG AAG AGG CTG CTT TCA CCG
AAT CTG GCG AAG CAG TCA ACT CTG CGAA

Sequence No.: 32

Length: 100

Type: DNA

Microorganism: Corynebacterium glutamicum

Category: Valyl tRNA-synthase

Sequence:

ACC TGG CTA ACG CAC AGA AGG AAT TGG AAA CCA CCG
CAA AGA AGC TGG GTA ATG AGG CTT TCC TGT CCA AGG
CAC CTG ATG CAG TGG TAG ACA AGA TCCG

Sequence No.: 33

Length: 100

Type: DNA

Microorganism: Mycobacterium tuberculosis

Category: Phenylalanyl tRNA-synthase beta subunit

Sequence:

CGA GGA GCT CAC CGG CTA CAA GAA GCC GAT CCG GGC
CTG CGC GGT AGA TAT CGG CGA TCG GCA GTA TCG CGA
GAT TAT TTG TGG TGC AAC CAA TTT CGCG

Sequence No.: 34

Length: 100

Type: DNA

Microorganism: Mycobacterium tuberculosis

Category: Histidyl tRNA-synthase

Sequence:

GAG GCC GGG ACG GTC GCA GTG AAG GAC TTG ACG ACG
GGT GAG CAA GTT TCG GTC TCG ATG GAT TCG GTT GTG
GCC GAA GTA ATT TCG CGG CTG GCT GGGT

Sequence No.: 35

Length: 100

Type: DNA

Microorganism: Mycobacterium tuberculosis

Category: Cysteinyl tRNA-synthase

Sequence:

AAG CGA TTG CTG AAA TGG TCG AGC TCA TCG AAA AAA
TGC TGG CGT GCG GGG CGG CCT ATG TCA TAG ACC GGG
AAA TGG GAG AGT ACC AGG ACA TCT ACTT

Sequence No.: 36

Length: 100

Type: DNA

Microorganism: Mycobacterium tuberculosis

Category: Leucyl tRNA-synthase

Sequence:

TCG TGG AAG TAA TTG CCG GCG GCA ATA TTT CGG AAT
CCG CGT ATA CAG GCG ATG GCA TCC TGG TCA ACT CGG
ATT ACC TCA ATG GAA TGA GCG TGC CAGC

Sequence No.: 37

Length: 100

Type: DNA

Microorganism: Pseudomonas aeruginosa

Category: Glutamyl and Glutaminyl tRNA-synthase

Sequence:

TCC TGG CTG AAC GGG CAG TGG ATC CGC GAG CAG TCC
GTG GAG GAG TTC GCC CGT GAG GTG CAG AAG TGG GCG
TTG AAT CCC GAA TAC CTG ATG AAG ATCG

Sequence No.: 38

Length: 100

Type: DNA

Microorganism: Pseudomonas aeruginosa

Category: Phenylalanyl tRNA-synthase beta subunit

Sequence:

AGC CCT GGA ACT AAC GGT GGT AGC CGA CGG GGA AGG
GCA GTG GTC GGT CGG CGT ACC GAG CCA TCG CTT CGA
CAT TTC CCT GGA AGT CGA CCT GAT CGAG

Sequence No.: 39

Length: 100

Type: DNA

Microorganism: Pseudomonas aeruginosa

Category: Histidyl tRNA-synthase

Sequence:

TGG TTG GCG CGC CGA CCC TGC ACG ACT ATC TCG ACG
AAG AAT CGA TCG CCC ACT TCG AGG GTC TCA AGG CCC
GCC TGG ATG CTG TCG GAC TTC GCT ACGA

Sequence No.: 40

Length: 100

Type: DNA

Microorganism: Pseudomonas aeruginosa

Category: Methionyl tRNA-synthase

Sequence:

TTC GAG CCC CTG ATG ACC CGT ATC GAG CCC GCG AAA
GTA GAA GCC ATG ATC GAA GCC TCC AAG GAA GAC CTC
GCC GCC GCA TCC CAA CCC GCC GGC AACG

Sequence No.: 41

Length: 100

Type: DNA

Microorganism: Pseudomonas aeruginosa

Category: Threonyl tRNA-synthase

Sequence:

CGA TTT CAT CAA GCT GAC CTT GCA GGT CTA CCG CGA
CTT CGG TTT CAC CGA CAT CGC CAT GAA GCT GTC GAC
CCG TCC GGC CAA GCG CGT CGG TTC CGAC

Sequence No.: 42

Length: 100

Type: DNA

Microorganism: Pseudomonas aeruginosa

Category: Prolyl tRNA-synthase

Sequence:

CGA AGG CGT CCT GGA ATA CAA GGG CCG CCG CGA CAG
CGA GTC GCA GAA CCT GCC GAT CGG CGA ACT GAT GTC
GTT CAT CAC CGA AAA ACT CAG CCG CTGA

Sequence No.: 43

Length: 100

Type: DNA

Microorganism: Pseudomonas aeruginosa

Category: Leucyl tRNA-synthase

Sequence:

ATG CAC GAG CAA TAT CAG CCA CTC GAG ATC GAA ACC
CAG GCC CAG AAC TAC TGG AAA GAA CAC CAG TCC TTC
CTC GTC AGG GAG CTA CCC GAC AAG GAGA

Sequence No.: 44

Length: 100

Type: DNA

Microorganism: Xylella fastidiosa

Category: Glutamyl and Glutaminyl tRNA-synthase

Sequence:

CCG AGC CCA GCC TGA GAA GAA AGA CTT TAT CCG GCA
GAT CAT CAG AGA AGA CTT AGC CCA CGG CAC ACA CAC
TCA CAT CCG CAC CCG TTT CCC CCC AGAA

Sequence No.: 45

Length: 100

Type: DNA

Microorganism: Xylella fastidiosa

Category: Alanyl tRNA-synthase

Sequence:

CGT GAG GTT GGC GCG TTT ACA GTG CCA GCC TAC TGT
TAA CTC CAT GAA CAT ACC TAC CAA ATT CAC CAC TTC
AAA GAT TCG TAG CGA TTT TCT AGA ATTT

Sequence No.: 46

Length: 100

Type: DNA

Microorganism: Xylella fastidiosa

Category: Phenylalanyl tRNA-synthase alpha subunit

Sequence:

GCA GAA CTT AGA TCA TTT GGA AGT TTT GCG CGT AGC
GCT TCT TGG TAA AAA TGG CAG CAT CAC CTT GCA GCT
CAA GCA ACT TGG TAA ATT GCC AGT TGAG

Sequence No.: 47

Length: 100

Type: DNA

Microorganism: Xylella fastidiosa

Category: Arginyl tRNA-synthase

Sequence:

ATG CTA ACG CGC TTC TCA TAC AAA CGG TCA GAT AAA
ATT ACG CTA TCT ATC GCC ACA CAC CCA CAC CCA CAC
GTG AAA GCC CCA CTC CGC GCC CTG ATTT

Sequence No.: 48

Length: 100

Type: DNA

Microorganism: Xylella fastidiosa

Category: Isoleucyl tRNA-synthase

Sequence:

CGG TGC CGT CCA CAC CGC CCC TGG CCA CGG CCA AGA
AGA CCA CCA AGT CTT TCA ACA ATA CGG ATT ACT CAA
TCA CTA CAG CGC TGC CGA ACT CAA CCCC

Sequence No.: 49

Length: 100

Type: DNA

Microorganism: Xylella fastidiosa

Category: Phenylalanyl tRNA-synthase beta subunit

Sequence:

TGG AGC GAA CTG GCA ATC ACC GTG CGC GAT GTC ATT
GGA TCG TTA CTT CGT GAC GTG AAA CTA TTT GAC CGC
TAC GTC GGT AAG GGT ATC GAA CCA GGCT

Sequence No.: 50

Length: 100

Type: DNA

Microorganism: Xylella fastidiosa

Category: Histidyl tRNA-synthase

Sequence:

TTG CCA CCC GTT CCA CCA GCC ATG CCG ATG CAT TGG
TGC GCC TGG ATG CAC TGA TCG AAG ATT CAG GGC CAG
AGG CAC ACG AGA TGC TGC GCC AAG GTGT

Sequence No.: 51

Length: 100

Type: DNA

Microorganism: Xylella fastidiosa

Category: Methionyl tRNA-synthase

Sequence:

TCG GTG GCC ACA CCA TCC ACC CCT ACT CCC CCT TAT
TCA CCC GCA TCG ATA AAA AAG TCA TTG AAG TGA TGA
TCA ACG CCT CCA AAG ACA CCC TCG CCCC

Sequence No.: 52

Length: 100

Type: DNA

Microorganism: Xylella fastidiosa

Category: Tyrosyl tRNA-synthase

Sequence:

TAA GCC ACT TAG TCA TGA GGA TGT ATT GGC TAA TGC
CCG TAC CTA CGA GGA TCA GGT ATT TAA GGT TTT GGA
TCG TAC CCT TAC CGA GGT GAG GTT CAAT

Sequence No.: 53

Length: 100

Type: DNA

Microorganism: Xylella fastidiosa

Category: Seryl tRNA-synthase

Sequence:

GAA GAG GAC ATG TTC AGT ACT CAG CTC GGT GAG CAT
CGG CGC TAT TTG ATT TCG ACC TCT GAA ATT TCG CTG
ACT AAT TTA GTA CGT AAC GAG ATT ATTG

Sequence No.: 54

Length: 100

Type: DNA

Microorganism: Xylella fastidiosa

Category: Aspartyl tRNA-synthase

Sequence:

TGA CGG TGG ATT CGC GTG CCA TCG ACC AGA ACA ACT
CAG AAC TGT TCA AGG TTG CTA GTG GCT TGA GTT ATG
AGG ATG TAC TAC AAG TTG AAG GCG TCGT

Sequence No.: 55

Length: 100

Type: DNA

Microorganism: Xylella fastidiosa

Category: Tryptophanyl tRNA-synthase

Sequence:

GGA AGG GGA TTC TTA ATC GTG CGC ATG CTT ATA AGG
CGG TGG TGG ATA AGA ATC GTA GTG AGG GGG GGG ATG
ATG ATG CCG GGA TCA CGG CCG GTC TGTT

Sequence No.: 56

Length: 100

Type: DNA

Microorganism: Xylella fastidiosa

Category: Cysteinyl tRNA-synthase

Sequence:

GTG CAT GGG TTG GTG GCA AAG CAT CCT CCC GAA GCA
CTA CGT TAC GCG CTG CTG TCG GCA CAT TAT CGG AAA
CCG CTG GAC TGG TCA GAG GCA CTC ATCG

Sequence No.: 57

Length: 100

Type: DNA

Microorganism: Xylella fastidiosa

Category: Threonyl tRNA-synthase

Sequence:

AGC ATG ACG CCA TGC TGC GAA TCA TCA CTC CTG AGG
ATC AAG AAG CTC TGG ACA TCA TAC GTC ACT CTT GTG
CTC ACT TGG TTG GCC ATG CGG TCA AGCA

Sequence No.: 58

Length: 100

Type: DNA

Microorganism: Xylella fastidiosa

Category: Prolyl tRNA-synthase

Sequence:

ACT CGT TCC ATC TGA ACG ATG CTG ATT TGG TAC GTG
AGT ACG AAA ATA TGA GGG CGA CGT ATA CAC GTA TCT
TTA CCA GGC TGG GGT TGG AGT TCC GTGC

Sequence No.: 59

Length: 100

Type: DNA

Microorganism: Xylella fastidiosa

Category: Leucyl tRNA-synthase

Sequence:

AAA TAC GGT TTA CCC ATC CGT CAA GTG ATC GCA CTC
AAA GAA CCT AAA AAC CAA GAC GAA TCC ACA TGG GAA
GGA GAT GTC TGG GGT GAT TGG TAC GCTG

Sequence No.: 60

Length: 100

Type: DNA

Microorganism: Xylella fastidiosa

Category: Valyl tRNA-synthase

Sequence:

GGA AGG GTT GAT CGA TGT GGA TGC CGA GCG CGT GCG
CCT GGA TAA AGA AAT CAA ACG TGT CGA AAG CGA AAT
CGA TAA AAG CAA CGG GAA ATT GAG TAAC

1. Method to detect and identify microorganisms that are present in a sample, wherein said method comprises hybridizing total DNA isolated from said sample with DNA fragments from regions codifying for tRNA synthases (hereinafter referred simply as tRNA-synthases), selected for their specificity for each taxon to be detected and identified.

2. Method according to claim 1, wherein said selected tRNA-synthase fragments are designed by comparing sequences that codify for tRNA-synthases of the relevant taxon with a database containing gene sequences codifying for the same tRNA-synthase in other known taxons.

3. Method according to claim 2, wherein said comparison is performed by using a public database.

4. Method according to claim 2, wherein said comparison is performed by using a database of tRNA-synthase sequences built from a public database.

5. Method according to claim 2, wherein a second selection round on the selected tRNA-synthase fragments based on the thermodynamic stability of said selected tRNA-synthase fragments is carried out.

6. Method according to claim 1, wherein said taxons for which tRNA-synthase fragments are being selected, are chosen from: Acinetobacter spp.; Aeropyrum pernix; Agrobacterium tumefaciens, Anabaena spp.; Anaplasma marginale; Aquifex aeolicus, Archaeoglobus fulgidus; Azoarcus spp.; Bacillus anthracis; Bacillus cereus; Bacillus clausii, Bacillus halodurans; Bacillus licheniformis, Bacillus subtilis, Bacillus thuringiensis; Bacteroides fragilis; Bacteroides thetaiotaomicron; Bartonella henselae; Barionella quintana; Bdellovibrio bacieriovorus; Bifidobacterium longum; Blochmannia floridanus; Bordetella bronchiseptica; Bordetella parapertussis; Bordetella pertussis; Borrelia burgdorferi; Borrelia garinii; Bradyrhizobium japonicum; Brucella abortus; Brucella melitensis; Brucella suis; Buchnera aphidicola; Burkholderia mallei; Burkholderia pseudomallei; Campylobacter jejuni; Caulobacter crescentus; Chlamydia muridarum (Chlamydia trachomatis MoPn); Chlamydia trachomatis, Chlamydophila abortus; Chlamydophila caviae; Chlamydophila pneumoniae; Chlorobium tepidum; Chromobacterium violaceum; Clostridium acetobutylicum; Clostridium perfringens; Clostridium tetani; Corynebacterium diphtheriae gravis; Corynebacterium efficiens; Corynebacterium glutamicum; Coxiella burnetii; Dehalococcoides ethenogenes; Deinococcus radiodurans; Desulfotalea psychrophila; Desulfovibrio vulgaris; Ehrlichia ruminantium, Enterococcus faecalis; Erwinia carotovora atroseptica; Escherichia coli; Francisella tularensis; Fusobacterium nucleatum; Geobacillus kaustophilus, Geobacter sulfurreducens; Gloeobacter violaceus; Gluconobacter oxydans; Haemophilus ducreyi; Haemophilus influenzae; Haloarcula marismortui; Halobacterium sp.; Helicobacter hepaticus, Helicobacter pylori; Idiomarina loihiensis; Lactobacillus acidophilus; Lactobacillus johnsonii; Lactobacillus plantarum; Lactococcus lactis subsp. lactis; Legionella pneumophila; Leifsonia xyli xyli; Leptospira interrogans; Leptospira interrogans; Listeria innocua; Listeria monocytogenes, Listeria monocytogenes; Mannheimia succiniciproducens; Mesoplasma florum; Mesorhizobium loti; Methanobacterium thermoautotrophicum; Methanococcus jannaschii; Methanococcus maripaludis; Methanopyrus kandleri; Methanosarcina acetivorans, Methanosarcina mazei; Methylococcus capsulatus; Mycobacterium avium paratuberculosis; Mycobacterium bovis bovis; Mycobacterium leprae, Mycobacterium tuberculosis; Mycoplasma gallisepticum; Mycoplasma genitalium, Mycoplasma hyopneumoniae; Mycoplasma mobile, Mycoplasma mycoides mycoides; Mycoplasma penetrans; Mycoplasma pneumoniae; Mycoplasma pulmonis; Nanoarchaeum equitans; Neisseria gonorrhoeae; Neisseria meningitidis; Nitrosomonas europaea; Nocardia farcinica; Oceanobacillus iheyensis; Parachlamydia sp.; Pasteurella multocida; Photobacterium profundum; Photorhabdus luminescens laumondii; Phytoplasma spp.; Picrophilus torridus; Porphyromonas gingivalis; Prochlorococcus marinus; Propionibacterium acnes; Pseudomonas aeruginosa; Pseudomonas putida, Pseudomonas syringae; Pyrobaculum aerophilum; Pyrococcus abyssi; Pyrococcus furiosus; Pyrococcus horikoshii; Ralstonia solanacearum; Rhodopirellula baltica; Rhodopseudomonas palustris; Rickettsia conorni; Rickettsia prowazekii; Rickettsia typhi; Salmonella enterica; Salmonella typhi; Salmonella typhimurium; Shewanella oneidensis, Shigella flexneri; Silicibacter pomeroyi; Sinorhizobium meliloti; Staphylococcus aureus; Staphylococcus epidermidis; Streptococcus agalactiae; Streptococcus mutans; Streptococcus pneumoniae; Streptococcus pyogenes; Streptococcus thermophilus; Streptomyces avermitilis; Streptomyces coelicolor; Sulfolobus solfataricus; Sulfolobus tokodaii; Symbiobacterium thermophilum; Synechococcus spp.; Synechocystis spp.; Thermoanaerobacter tengcongensis; Thermococcus kodakaraensis; Thermoplasma acidophilum; Thermoplasma volcanium; Thermosynechococcus elongatus; Thermotoga maritima; Thermus thermophilus; Treponema denticola; Treponema pallidum, Tropheryma whipplei; Ureaplasma urealyticum, Vibrio cholerae; Vibrio fischeri; Vibrio parahaemolyticus; Vibrio vulnificus; Vibrio vulnificus, Wigglesworthia brevipalpis; Wolbachia spp.; Wolinella succinogenes; Xanthomonas axonopodis; Xanthomonas campestris, Xanthomonas oryzae; Xylella fastidiosa, Yersinia pestis; Yersinia pestis; Yersinia pseudotuberculosis and Zymomonas mobilis.

7. Method according to claim 1, wherein said method comprises sinthesizing tRNA-synthase fragments specific for each taxon and using said fragments in molecular biology techniques for the detection and identification of microorganisms.

8. Method according to claim 7, wherein said molecular biology techniques for the detection and identification of microorganisms are selected from fluorescent in situ hybridization (FISH), denaturing gradient gel electrophoresis or temperature gradient gel electrophoresis (DGGE, TGGE), conjugation with specific markers, such as detection probes, polymerase chain reaction (PCR), and macro and micro DNA arrays.

9. Method according to claim 8, wherein said molecular biology technique for the detection and identification of microorganisms are preferably DNA arrays.

10. Method according to claim 9, wherein an array that comprises at least one tRNA-synthase fragment that specifically identifies one of the taxons to be detected is manufactured.

11. Array for the detection and identification of microorganisms, wherein said array comprises at least one tRNA-synthase fragment specific for each taxon to be detected and identified bound to its surface, said tRNA-synthase fragment having been designed according to the method described in claim 1.

12. Array according to claim 11, wherein taxons to be identified are selected from: Acinelobacter spp.; Aeropyrum pernix; Agrobacterium tumefaciens; Anabaena spp.; Anaplasma marginale; Aquifex aeolicus; Archaeoglobus fulgidus; Azoarcus spp.; Bacillus anthracis; Bacillus cereus; Bacillus clausii; Bacillus halodurans; Bacillus licheniformis; Bacillus subtilis, Bacillus thuringiensis; Bacteroides fragilis; Bacteroides thetaiotaomicron; Bartonella henselae; Bartonella quintana; Bdellovibrio bacteriovorus; Bifidobacterium longum; Blochmannia floridanus, Bordetella bronchiseptica, Bordetella parapertussis; Bordetella pertussis; Borrelia burgdorferi, Borrelia garinii; Bradyrhizobium japonicum, Brucella abortus; Brucella melitensis; Brucella suis; Buchnera aphidicola; Burkholderia mallei; Burkholderia pseudomallei, Campylobacter jejuni; Caulobacter crescentus; Chlamydia muridarum (Chlamydia trachomatis MoPn); Chlamydia trachomatis; Chlamydophila abortus, Chlamydophila caviae; Chlamydophila pneumoniae; Chlorobium tepidum; Chromobacterium violaceum; Clostridium acetobutylicum; Clostridium perfringens; Clostridium tetani; Corynebacterium diphtheriae gravis; Corynebacterium efficiens; Corynebacterium glutamicum; Coxiella burnetii; Dehalococcoides ethenogenes; Deinococcus radiodurans; Desulfotalea psychrophila; Desulfovibrio vulgaris; Ehrlichia ruminantium; Enterococcus faecalis; Erwinia carotovora atroseptica; Escherichia coli; Francisella tularensis; Fusobacterium nucleatum; Geobacillus kaustophilus; Geobacter sulfurreducens; Gloeobacter violaceus; Gluconobacter oxydans; Haemophilus ducreyi; Haemophilus influenzae; Haloarcula marismortui, Halobacterium sp.; Helicobacter hepaticus; Helicobacter pylori; Idiomarina loihiensis; Lactobacillus acidophilus; Lactobacillus johnsonii; Lactobacillus plantarum, Lactococcus lactis subsp. lactis; Legionella pneumophila, Leifsonia xyli xyli; Leptospira interrogans, Leptospira interrogans; Listeria innocua; Listeria monocytogenes; Listeria monocytogenes; Mannheimia succiniciproducens; Mesoplasma florum; Mesorhizobium loti; Methanobacterium thermoautotrophicum; Methanococcus jannaschii; Methanococcus maripaludis; Methanopyrus kandleri; Methanosarcina acetivorans, Methanosarcina mazei; Methylococcus capsulatus; Mycobacterium avium paratuberculosis; Mycobacterium bovis bovis; Mycobacterium leprae; Mycobacterium tuberculosis; Mycoplasma gallisepticum; Mycoplasma genitalium; Mycoplasma hyopneumoniae; Mycoplasma mobile; Mycoplasma mycoides mycoides; Mycoplasma penetrans; Mycoplasma pneumoniae; Mycoplasma pulmonis; Nanoarchaeum equitans; Neisseria gonorrhoeae; Neisseria meningitidis; Nitrosomonas europaea; Nocardia farcinica; Oceanobacillus iheyensis; Parachlamydia sp.; Pasteurella multocida; Photobacterium profundum; Photorhabdus luminescens laumondii, Phytoplasma spp.; Picrophilus torridus, Porphyromonas gingivalis; Prochlorococcus marinus; Propionibacterium acnes; Pseudomonas aeruginosa; Pseudomonas putida; Pseudomonas syringae; Pyrobaculum aerophilum; Pyrococcus abyssi; Pyrococcus furiosus; Pyrococcus horikoshii; Ralstonia solanacearum; Rhodopirellula baltica; Rhodopseudomonas palustris; Rickettsia conorni; Rickettsia prowazekii; Rickettsia typhi; Salmonella enterica; Salmonella typhi; Salmonella typhimurium; Shewanella oneidensis; Shigella flexneri; Silicibacter pomeroyi; Sinorhizobium meliloti; Staphylococcus aureus; Staphylococcus epidermidis; Streptococcus agalactiae; Streptococcus mutans; Streptococcus pneumoniae; Streptococcus pyogenes; Streptococcus thermophilus; Streptomyces avermitilis; Streptomyces coelicolor, Sulfolobus solfataricus; Sulfolobus tokodaii; Symbiobacterium thermophilum, Synechococcus spp.; Synechocystis spp., Thermoanaerobacter tengcongensis; Thermococcus kodakaraensis; Thermoplasma acidophilum; Thermoplasma volcanium; Thermosynechococcus elongatus; Thermotoga maritima; Thermus thermophilus; Treponema denticola; Treponema pallidum; Tropheryma whipplei; Ureaplasma urealyticum; Vibrio cholerae; Vibrio fischeri; Vibrio parahaemolyticus; Vibrio vulnificus; Vibrio vulnificus; Wigglesworthia brevipalpis; Wolbachia spp.; Wolinella succinogenes; Xanthomonas axonopodis, Xanthomonas campestris; Xanthomonas oryzae; Xylella fastidiosa; Yersinia pestis; Yersinia pestis; Yersinia pseudotuberculosis and Zymomonas mobilis.

13. Array according to claim 12, wherein the tRNA-synthase fragment that allows the identification of Agrobacterium tumefaciens is selected from the tRNA-synthase fragments defined by SEQ ID NOS:1 to 17 and their respective reverse complementary sequences.

14. Array according to claim 13, wherein the tRNA-synthase fragments are either entire or contained in a larger sequence where specificity is given by said fragments, or are any of the sub-fragments contained therein.

15. Array according to claim 14, wherein said sub-fragments comprise preferably 50 to 70 nucleotides.

16. Array according to claim 12, wherein the tRNA-synthase fragment that allows the identification of Corynebacterium glutamicum is selected from the tRNA-synthase fragments defined by SEQ ID NOS:18 to 32 and their respective reverse complementary sequences.

17. Array according to claim 16, wherein the tRNA-synthase fragments are either entire or contained in a larger sequence where specificity is given by said fragments, or are any of the sub-fragments contained therein.

18. Array according to claim 17, wherein said sub-fragments comprise preferably 50 to 70 nucleotides.

19. Array according to claim 12, wherein the tRNA-synthase fragment that allows the identification of Mycobacterium tuberculosis is selected from the tRNA-synthase fragments defined by SEQ ID NOS:33 to 36 and their respective reverse complementary sequences.

20. Array according to claim 19, wherein the tRNA-synthase fragments are either entire or contained in a larger sequence where specificity is given by said fragments, or are any of the sub-fragments contained therein.

21. Array according to claim 20, wherein said sub-fragments comprise preferably 50 to 70 nucleotides.

22. Array according to claim 12, wherein the tRNA-synthase fragment that allows the identification of Pseudomonas aeruginosa is selected from the tRNA-synthase fragments defined by SEQ ID NOS:37 to 43 and their respective reverse complementary sequences.

23. Array according to claim 22, wherein the tRNA-synthase fragments are either entire or contained in a larger sequence where specificity is given by said fragments, or are any of the sub-fragments contained therein.

24. Array according to claim 23, wherein said sub-fragments comprise preferably 50 to 70 nucleotides.

25. Array according to claim 12, wherein the tRNA-synthase fragment that allows the identification of Xylella fastidiosa is selected from the tRNA-synthase fragments defined by SEQ ID NOS:44 to 60 and their respective reverse complementary sequences.

26. Array according to claim 25, wherein the tRNA-synthase fragments are either entire or contained in a larger sequence where specificity is given by said fragments, or are any of the sub-fragments contained therein.

27. Array according to claim 26, wherein said sub-fragments comprise preferably 50 to 70 nucleotides.
